(19) United States
(12) Patent Application Publication
(10) Pub. No.: US 2020/0360666 A1
(43) Pub. Date: Nov. 19, 2020

(54) GUIDE CATHETER WITH SUPPORT WIRES

(71) Applicant: Medtronic Vascular, Inc., Santa Rosa, CA (US)

(72) Inventors: David Killeen, Cork (IE); Suruchi Anand, Rohnert Park, CA (US); Brent Locsin, San Francisco, CA (US); Kelsey K. Kam, Santa Rosa, CA (US); Jaclyn Kawwas, San Francisco, CA (US); Kimaya N. Gupte, Santa Rosa, CA (US)

(21) Appl. No.: 15/930,085

(22) Filed: May 12, 2020

Related U.S. Application Data
(60) Provisional application No. 62/847,137, filed on May 13, 2019.

(51) Int. Cl. A61M 25/01; A61M 25/00

(52) U.S. Cl. CPC A61M 25/0147; A61M 2025/09008; A61M 25/0012

(57) ABSTRACT

In some examples, a catheter includes a hub assembly, an elongate member, and a plurality of support wires extending along a length of the elongate member. The plurality of support wires is configured to expand radially outward from the elongated body to help anchor a distal portion of the catheter within the vasculature the patient and provide a backup support to the elongate member, e.g., to reduce pushback on the catheter when a treatment device is advanced past the distal tip of the catheter.

This application claims the benefit of U.S. Provisional Application Ser. No. 62/847,137, entitled “GUIDE CATHETER WITH SUPPORT WIRES,” filed on May 13, 2019, the entire content of which is incorporated herein by reference.

### TECHNICAL FIELD

This disclosure relates to guide catheter systems for accessing intravascular target sites.

### BACKGROUND

Vasculature within the body of a patient may sometimes become occluded or narrowed by atherosclerotic plaque or other lesions along the vessel walls, which can result in reduced blood flow though the afflicted vessels. In the case of the coronary artery, these afflictions may be generally referred to as coronary heart disease or stenosis. In some cases, a clinician may deliver an interventional device (e.g., a stent or a balloon) to reduce the stenosis and expand the afflicted vessel to an increase blood flow. The interventional device may be delivered using a percutaneous transcatheter (or transluminal) technique, during which a clinician may use a guide catheter to gain access to the target site in the vasculature. The guide catheter may be introduced though an incision into a femoral (transfemoral) or radial (transradial) artery of a patient and advanced through the vasculature of the patient to gain access to the target treatment site. With the guide catheter in place, a treatment device such as a stent, a stent retriever, an embolic collection device, a balloon catheter, or the like may be introduced through the guide catheter and maneuvered/advanced through the vasculature to the target treatment site at or near the stenosis of the diseased vessel.

### SUMMARY

This disclosure describes guide catheters with an elongated body that includes a plurality of wires attached to and extending along the length of the elongated body. The wires are configured to expand radially outward from the elongated body to help anchor and support the elongated body within the vasculature of a patient. In some examples, the wires may be used to provide a backup support to elongated body, thereby helping to seat the guide catheter within the vasculature of the patient and prevent any pushback on the guide catheter when a treatment device is advanced past the distal end of the guide catheter and further into the vasculature of the patient. While the design of the guide catheters described herein may be useful in a wide variety of treatment procedures, the design may be particularly useful in procedures where the guide catheter is introduced through the radial artery of a patient where, due to the angle at which the guide catheter approaches the target vessel, adequate backup support created by the native architecture of vasculature may be lacking or decreased relative to other access sites. In some other aspects, the disclosure also describes methods of delivering and using the guide catheters described herein.

In some examples, a catheter may include an elongate member and a plurality of support wires. The elongate member may extend along a longitudinal axis from a proximal end to a distal tip. The elongate member may define an inner lumen and a distal opening to the inner lumen. The plurality of support wires may extend along at least a distal portion of the elongate member. The plurality of support wires may be slidably engaged with a proximal constraint. A distal end of each support wire of the plurality of support wires may be attached to a distal constraint. The plurality of support wires may be configured to expand radially outwards between the proximal and distal constraints from a collapsed configuration to a deployed configuration. When in the deployed configuration, a section of at least one support wire of the plurality of support wires between the proximal and distal constraints may be configured to engage with a vessel wall to position the elongate member away from the vessel wall.

In some examples, a medical assembly may include a catheter and a treatment device. The catheter may include a hub assembly, an elongate member, and a plurality of support wires. The elongate member may extend along a longitudinal axis from a proximal end coupled to the hub assembly to a distal tip. The elongate member may define an inner lumen and a distal opening to the inner lumen. The plurality of support wires may extend along at least a distal portion of the elongate member. The plurality of support wires may be slidably engaged with a proximal constraint. A distal end of each support wire of the plurality of support wires may be attached to a distal constraint. The plurality of support wires may be configured to expand radially outwards between the proximal and distal constraints from a collapsed configuration to a deployed configuration. When in the deployed configuration, a section of at least one support wire of the plurality of support wires between the proximal and distal constraints may be configured to engage with a vessel wall in the deployed configuration. The treatment device may be configured to be received in the inner lumen of the elongate member.

In some examples, method of using a catheter may include advancing the catheter through vasculature of a patient. The catheter may include an elongate member and a plurality of support wires. The elongate member may extend along a longitudinal axis from a proximal end to a distal tip. The elongate member may define an inner lumen and a distal opening to the inner lumen. The plurality of support wires may extend along at least a distal portion of the elongate member. The plurality of support wires may be slidably engaged with a proximal constraint. A distal end of each support wire of the plurality of support wires may be attached to a distal constraint. The method also may include actuating the plurality of support wires to expand radially outwards between the proximal and distal constraints from a collapsed configuration to a deployed configuration. When in the deployed configuration, a section of at least one support wire of the plurality of support wires between the proximal and distal constraints may be configured to engage with a vessel wall to position the elongate member away from the vessel wall. The method also may include advancing a treatment device through the inner lumen of the elongate member to a target treatment site.

The details of one or more examples are set forth in the accompanying drawings and the description below. Other features, objects, and advantages of examples according to this disclosure will be apparent from the description and drawings, and from the claims.

### BRIEF DESCRIPTION OF THE DRAWINGS

FIG. 1 is a conceptual diagram illustrating a side view of an example catheter that includes an elongate member extending from a proximal end to a distal tip.

FIGS. 2A and 2B are conceptual diagrams illustrating a side view and a cross sectional view, respectively, of the catheter illustrated in FIG. 1 with the support wires in a collapsed configuration.

FIGS. 3A and 3B are conceptual diagrams illustrating a side view and a cross sectional view, respectively, of the catheter illustrated in FIG. 1 with the support wires in a deployed configuration.

FIG. 4 is a conceptual diagram illustrating the catheter illustrated in FIG. 1 with the support wires in an deployed configuration engaging the vasculature of a patient.

FIGS. 5A and 5B are conceptual diagrams illustrating a side view and a cross sectional view, respectively, of a distal portion of an elongate member of an example catheter with support wires and a connecting member in a deployed configuration.

FIGS. 6A and 6B are conceptual diagrams illustrating a side view and a cross sectional view, respectively, of a distal portion of an elongate member of an example catheter with a spiral support wire in a deployed configuration.

FIG. 7 is a flow diagram illustrating an example method of delivering a treatment device to a target treatment site within a vasculature of a patient using an example catheter.

### DETAILED DESCRIPTION

This disclosure describes guide catheters (also referred to herein as “catheters”) and guide catheter systems for accessing intravascular target sites and introducing treatment devices at the target sites. In some examples, the target treatment site may be a site within the heart or vasculature of a patient that has a defect which is affecting blood flow. For example, the target treatment site may be a portion of a coronary artery such as the left coronary artery or other distal location that includes a calcified lesion, e.g., calcified plaque buildup, stenosis, aneurysm, or other diseased area. Various treatment devices and procedures, such as balloon angioplasty, stenting, thrombectomy, atherectomy, or other interventional procedures may be introduced into the vasculature of a patient and advanced to the treatment site containing the calcified lesion. The treatment devices may also be referred to as interventional devices in some examples. A treatment device can be navigated through the tortious vasculature of the patient using the aid of a guide catheter. The treatment device may be either housed within a lumen of the catheter or introduced through the catheter after the catheter is properly positioned within the vasculature of the patient.

The deployment of the treatment device from a distal opening (e.g., at a distal end or near a distal end) of the catheter used to deliver the device can often create an axial force in the opposite direction of deployment along the catheter. For example, when a treatment device is forced by a clinician distally relative to the catheter and further into the vasculature or lesion of the patient, the resistance encountered by the treatment device within the vessel can sometimes transfer the force into the catheter, thereby forcing the distal end of catheter in the proximal direction along its longitudinal axis rather than forcing the treatment device further into the vessel of the patient.

In some examples, this axial force exerted on the catheter in the proximal direction can cause the catheter to bend or become dislodged from its intended location within the vessel. For example, when attempting to pass through a stenosis, or when conducting a radial intervention using a guide catheter, the guide catheter may not have adequate backup support, and continued application of force to advance the treatment device through the stenosis may cause the distal end of the guide catheter to dislodge from the opening of the ostium of the coronary artery. As a result, the clinician may need to reposition the guide catheter in the coronary artery, which may increase the time required to perform the medical procedure.

Backup support may include support of a distal portion of a guide catheter (or other medical device, such as another catheter or a guidewire) using internal surfaces of the vasculature of a patient. Backup support on a catheter may help combat the axial force that may dislodge the guide catheter from its intended location in a vessel. In some medical procedures, the natural curvature of the vasculature of the patient can help produce some degree of backup support. However, other medical procedures, such as accessing a coronary artery from a right radial approach, may lack a sufficient number of turns and natural backup support options to establish a sufficient amount of backup support for the catheter from the anatomy of the patient. Additionally, or alternatively, particular types of lesions such as calcified lesions may be particularly resistive to the advancement of a treatment device by the application of a pushing force along a longitudinal axis of the catheter. Additionally, or alternatively, deep seating the guide catheter, e.g., pushing the guide catheter further distally into the coronary artery, may reduce dislodgment, but may increase risk of the guide catheter occluding the coronary artery or interfering with blood flow to the coronary artery.

The catheters described herein include a plurality of support wires that extend along the exterior surface of a distal portion of the catheter. The wires are configured to expand radially outward from the catheter in order to contact the surrounding vessel wall to establish additional backup support for the catheter. The wires may increase a contact area with the vessel wall, which may help provide the additional backup support for the catheter. The additional backup support may help prevent the movement of the catheter within the vasculature of the patient, particularly during the advancement of treatment device into a targeted lesion or other treatment site, and increase stability of the guide catheter during the advancement of the treatment device through the guide catheter. In addition, selective expansion of the wires away from the guide catheter may help modify the position of the guide catheter in a vessel relative to a center of the vessel, e.g., to re-center the catheter in the vessel, which may also enable easier delivery of an inner catheter or other device through a lumen of the guide catheter.

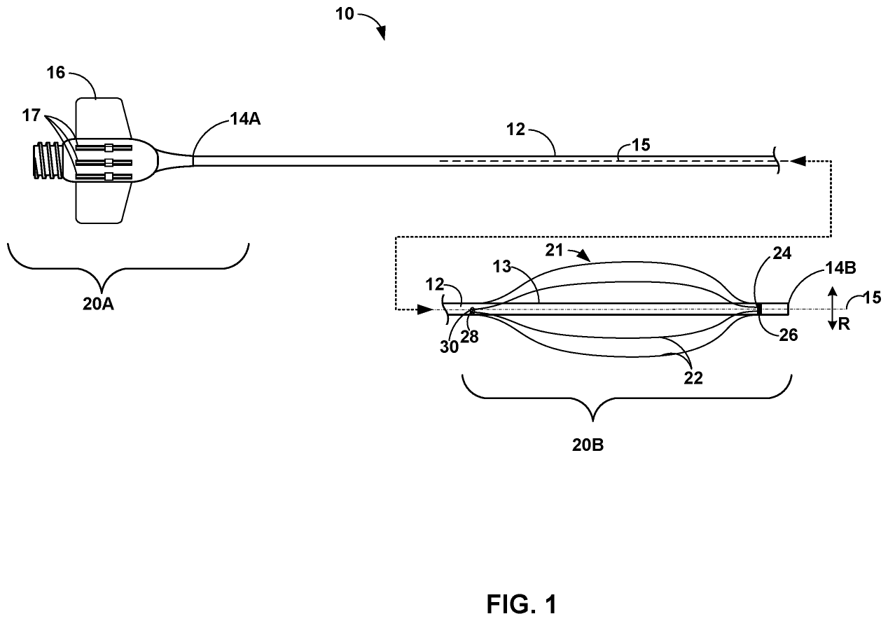

FIG. 1 is a conceptual diagram illustrating a side view of an example catheter 10, which includes an elongate member 12 extending from proximal end 14A to distal tip 14B. The side view of catheter 10 illustrates in FIG. 1 illustrates catheter 10 along a central longitudinal axis 15 of elongate member 12. Catheter 10 may include a hub assembly 16 connected to proximal end 14A of elongate member 12. In some examples, proximal end 14A may extend into hub assembly. Hub assembly 16, including proximal end 14A of elongate member 12, forms part of a proximal portion 20A of catheter 10. Catheter 10 also includes a distal portion 20B that includes distal tip 14B of elongate member 12. The designations of proximal portion 20A and distal portion 20B are used to describe different regions of catheter 10 (as divided along a length of catheter 10) and may be of any suitable length. In some examples, elongate member 12 may also be characterized as having one or more intermediate portions separating proximal portion 20A and distal portion 20B.

Catheter 10 includes a plurality of support wires 22 (“support wires 22”) that extend along longitudinal axis 15. Catheter 10 may include any suitable number of support wires 22. As illustrated in FIG. 1, catheter 10 includes four support wires 22. In other examples, catheter 10 may include a fewer number of support wires 22, such as two or three support wires 22, or a greater number of support sires 22, such as five or more support wires 22. Support wires 22 may have any suitable configuration, such as, but not limited to, flat or round cross-sections (the cross-section being taken in a direction orthogonal to a longitudinal axis of the respective wire), and can be formed from any suitable material, such as, but not limited to, a relatively flexible metal or polymeric material, or combinations thereof.

Each of support wires 22 may be constrained at a distal constraint 24 and a proximal constraint 28. Distal constraint 24 and proximal constraint 28 are configured to control where along distal portion 20B of elongate member 12 support wires 22 can expand radially outward (e.g., in direction “R”) relative to central longitudinal axis 15. In some examples, each of the support wires 22 is fixed at distal constraint 24 and slidably engages with proximal constraint 28.

In this way, at least a portion of support wires 22 may extend along exterior surface 13 of elongate member 12, such that actuation of each of support wires 22 in the distal direction causes each of support wires 22 to expand radially outward between distal constraint 24 and proximal constraint 28.

In some examples, each of support wires 22 includes a distal end which may be attached to elongate member 12 distal constraint 24. Within distal portion 20B of elongate member 12, a distal portion 21 of each of support wires 22 (e.g., the portion of support wires 22 shown in FIG. 1) is positioned adjacent to exterior surface 13 of elongated member 12 but is separated from the elongate member 12, thereby allowing support wires 22 to be expanded in the radial direction, “R”.

Distal constraint 24 may fix the distal ends of support wires 22 to elongate member 12 using any suitable technique. For example, distal constraint 24 may include a marker band 26 in some examples. In some examples, marker band 26 may include a radiopaque marker band. In some examples, distal constraint 24 may include adhering or mechanically fastening the distal ends of support wire 22 to marker band 26. Additionally, or alternatively, distal constraint 24 may include other structures within or connected to elongated member 12. In some examples, distal ends of support wires 22 may be bonded to elongate member 12, such as a distal most polymer/braided section of elongate member 12. In this way, bonding support wires 22 to elongate member 12 may provide a more robust adhesion method compared to support wires 22 sit into a distal end of elongate member 12. In some examples, distal ends of support wire 22 may be embedded within the body of elongate member 12 or have an outer jacket of material (e.g., a polymeric jacket) secured over distal ends of support wire 22. Having distal ends of support wires 22 embedded within the body or secured beneath an outer jacket may help keep exterior surface 13 of elongate member 12 smooth where distal ends of support wires 22 are secured to elongate member 12. The smoothness of the exterior surface may improve the navigability of distal tip 14B through the vasculature of the patient by reducing any resistance generated between the inner wall of the vessel and distal ends of support wires 22.

In some examples, distal ends of support wires 22 and/or distal constraint 24 may be within a range from about 1 millimeter to about 10 centimeters proximal to distal tip 14B of elongate member 12. The position of distal constraint 24 may be selected to provide backup support at a selected anatomical structure. For example, the selected anatomical structure may include a selected position in the vasculature of the patient, such as the ascending aorta. The distance from the selected anatomical structure of a target treatment site may be used to determine the position of distal constraint 24. In some examples, distal constraint 24 is movable relative to elongate member 12 and the position of distal constraint 24 may be selected by a clinician by, for example, manually adjusting a position of a marker band 26 or other structure configured to fix distal constraint 24 to elongate member 12. The clinician may then subsequently secure marker band 26 in place relative to elongate member 12 for the medical procedure. In other examples, however, distal constraint 24 is not movable relative to elongate member 12 and may not be moved relative to elongate member 12 without adversely impacting the structural integrity of elongate member 12.

A proximal end of the distal portion 21 of support wires 22 may be constrained on exterior surface 13 at a proximal constraint 28. Proximal constraint 28 may include a structure configured to slidably engage support wires 22. For example, elongate member 12 may include one or more lumens (not shown) extending from proximal portion 20A to a distal opening 30 at distal portion 20B. In some examples, the one or more lumens may be defined by a wall of elongate member 12. Support wires 22 may extend within the one or more lumens from a proximal end of support wires 22 (not shown), e.g., operatively coupled to hub assembly 16 or another actuation mechanism, protrude out of distal opening 30, and extend to distal constraint 24.

In some examples, support wires 22 may be individually controllable at hub assembly 16 to expand the respective support wire 22 (e.g., a portion of the wire 22) radially outward relative to central longitudinal axis 15. For example, the proximal end of support wires 22 may be mechanically coupled to a single wire (e.g., a pushable and/or pullable wire or other elongated support element) extending through the one or more lumens of elongate member 12 from hub assembly 16 to, or nearly to, distal opening 30. By individually controlling support wires 22, a clinician may push an individual support wire 22 in a distal direction to cause the support wire 22 to expand radially outward, pull an individual support wire 22 in a proximal direction to cause the support wire 22 to collapse radially inward, or both.

In this way, a mechanical push system, e.g., hub assembly 16, may be configured to actuate support wires 22 to push and/or pull support wires 22 in a distal-proximal direction between an collapsed configuration and a deployed configuration. For example, hub assembly may include one or more control member 17. Each of control members 17 may be operatively coupled one or more support wires 22. Control member 17 may include a slidable lever or other feature that may be manipulated by an clinician to control support wires 22 between the collapsed configuration and the deployed configuration. In this way, hub assembly 16 may not require electrical components or bulky handle modules to actuate support wires 22, which may help reduce the cost of catheter 10 and enable catheter 10 to be relatively user friendly.

In some examples, support wires 22 may be controllable at hub assembly 16 as one or more groups of support wires 22. For example, the proximal end of two or more distal support wires 22 may be coupled to a single proximal wire (e.g., a pushable and/or pullable wire or other elongated support element) extending through the one or more lumens of elongate member 12 from hub assembly 16 to, or nearly to, distal opening 30. By controlling groups of support wires 22 with a common proximal wire, a clinician may efficiently push a group of support wires 22 in a distal direction to cause the group of support wires 22 to expand radially outward, efficiently pull a group of support wire 22 in a proximal direction to cause the group of support wire 22 to collapse radially inward, or both. In some examples, actuating a group of support wires 22 using a single proximal push/pull wire may be relatively easier, e.g., less friction or fewer control members, compared to actuating each of support wires 22 individually. Additionally, or alternatively, controlling groups of support wires 22 with proximal push/pull wires requires fewer wires to extend along proximal portion 20A (or an intermediate portion) of elongate member 12. Fewer wires extending along proximal portion 20A (or an intermediate portion) of elongate member 12 may increase the flexibility of elongate member 12 compared to a catheter in which each support wire 22 extends from proximal end 14A to distal constraint 24. In some examples, controlling a group of support wires 22 may enable a clinician to expand support wires 22 on a first side of elongate member 12 and, subsequently, expand the other support wires 22 on a second side of elongate member 12.

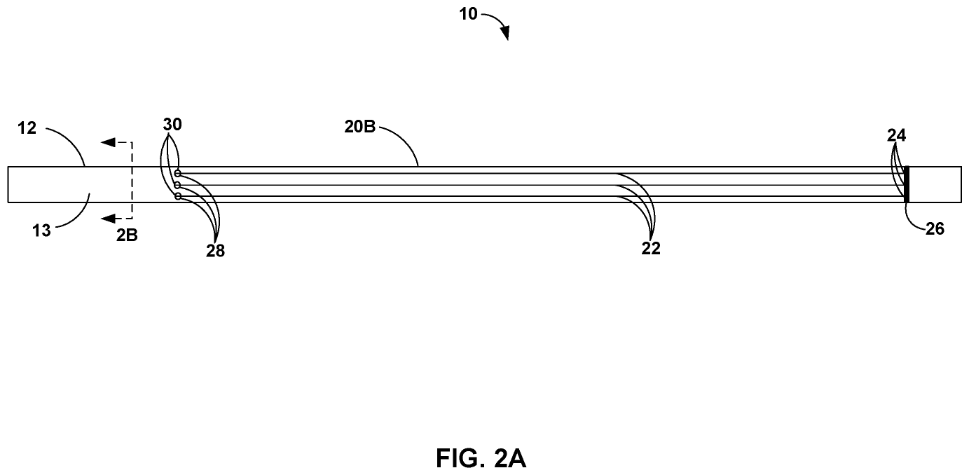

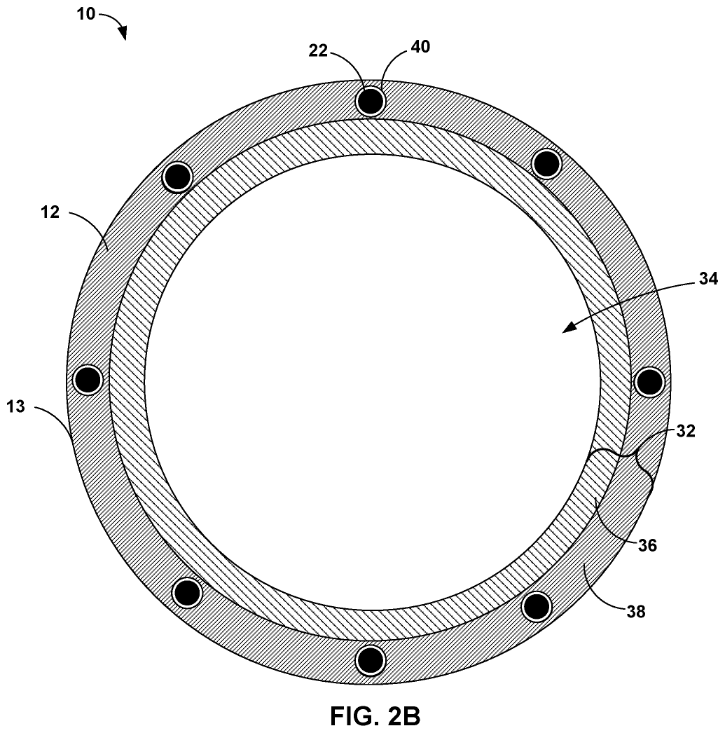

FIGS. 2A-4 are conceptual diagrams illustrating additional views of the distal portion 20B of catheter 10 showing various arrangement characteristics of support wires 22. FIG. 2A is a conceptual diagram illustrating distal portion 20B of elongate member 12 with support wires 22 in a collapsed configuration. When in the collapsed configuration, support wires 22 may have a lower profile compared to other support structures, such as, for example, a balloon support structure. As a result, when support wires 22 are in the collapsed configuration, catheter 10 may be in a relatively low profile configuration, which may facilitate the advancement of catheter 10 through vasculature of a patient. For example, when support wires 22 are in the collapsed configuration, the distal portion 21 of support wires 22 may be positioned on (e.g., in direct contact with or otherwise adjacent to) exterior surface 13 of distal portion 20B of elongate member 12. By positioning distal portion 21 of support wires 22 on exterior surface 13 of distal portion 20B, elongate member 12 and support wires 22 may define a relatively smooth surface when support wires 22 are in the collapsed configuration. The relatively smooth surface defined by exterior surface 13 and support wires 22 in the collapsed configuration may improve the navigability of distal tip 14B through the vasculature of the patient by reducing any resistance generated between the inner wall of the vessel and exterior surface 13 or support wires 22.

As illustrated in FIG. 2A, in some examples, support wires 22 are evenly distributed around an outer perimeter of distal portion 20B of elongate member 12. Substantially even distribution (e.g., even or near even distribution within limits of catheter manufacturing techniques) of support wires 22 may provide predictable contact points of support wires 22 with surrounding tissue when in the deployed configuration. For example, substantially even distribution of support wires 22 may have a substantially uniform distribution when expanded into the deployed configuration such that equal or nearly equal expansion of support wires 22 may result in catheter 10 being positioned at or near a center of a blood vessel of the patient. Additionally, or alternatively, substantially even distribution of support wires 22 may have the same or nearly the same orientation relative to the vasculature of the patient when introduced into the vasculature in any rotational orientation. This may reduce the skill required for a clinician to manipulate support wires 22 to provide backup support for catheter 10.

In other examples, support wires 22 may be unevenly distributed around an outer perimeter of distal portion 20B of elongate member 12. For example, a first set of support wires 22 may be spaced more closely together on a first side of elongate member 12 compared to a second set of support wires 22 on a second, opposing side of elongate member 12. Due to the higher density of the first set of support wires 22 spaced more closely together on a first side of elongate member 12, the first set of support wires 22 may provide a stiffer (e.g., more rigid) backup support compared to the second set of support wires 22 spaced further apart on the second side of elongate member 12 and enable additional support on the first side of elongate member 12. For example, the first side of elongate member 12 may have more support wires 22 to resist axial force on elongate member 12 compared to the second side of elongate member 12. By controlling, e.g., with hub assembly 16, a rotation of elongate member 12 relative to the vasculature of a patient, an clinician may orient a first set of relatively more closely spaced support wires 22 in a direction that may provide the backup support for catheter 10.

FIGS. 2B is a conceptual diagram illustrating a cross section (the cross-section taken along line 2B-2B shown in FIG. 2A in a direction orthogonal to longitudinal axis 15 (not shown)) of distal portion 20B of elongate member 12 with support wires 22 in an collapsed configuration. As shown in FIG. 2B, in some examples, elongate member 12 includes a wall 32 defining a lumen 34. Wall 32 may include any suitable construction, such as one or more coaxial polymer layers. For example, wall 32 may include interior layer 36 and exterior layer 38. Interior layer 36 may include any suitable polymer, a plurality of layers of one or more polymers, or one or more combinations of polymers, such as, for example, polytetrafluoroethylene, high-density polyethylene, polyether block amide, or a polymer configured to enable interventional devices, such as a balloon catheter, stent delivery system, or the like, to readily slide in lumen 34. Exterior layer 38 may include any suitable polymer, a plurality of layers of one or more polymers, or one or more combinations of polymers, such as, for example, polyethylene, polypropylene, polyether block amide or a polymer configured to provide a selected flexibility of elongate member 12. Interior layer 36 and exterior layer 38 may include any suitable shape and/or thickness, for example, a shape and/or a thickness of interior layer 36 and exterior layer 38 may be substantially uniform or vary circumferentially or along a length of elongate member 12.

Although not illustrated in FIG. 2B, in some examples, wall 32 may include one or more support structures, e.g., one or more coiled or braided structures. The coiled or braided structure may be formed from any suitable material, such as, but not limited to, a medical grade metal, nickel titanium alloy, or stainless steel. In some examples, the one or more support structures may be positioned between interior layer 36 and exterior layer 38, and/or embedded in one or both inner layer 36 and exterior layer 38.

Lumen 34 is sized to receive a selected treatment device. In some examples, the diameter of lumen 34 is not decreased as a result of support wires 22 because support wires 22 extend within wall 32 or along exterior surface 13 of elongate member 12. In some examples, the treatment device may include, but is not limited to, a balloon catheter, a stent delivery system, a thrombus removal system, an atherosclerosis removal system, or percutaneous coronary interventional systems. For example, the selected interventional treatment device may be either housed within lumen 34 of catheter 10 or introduced through lumen 34 after catheter 10 is properly positioned at a target treatment site within the vasculature of the patient. In some examples, the size of lumen 34 may be within a range from between about 4 French (Fr) to about 10 Fr, such as between about 5 Fr to about 8 Fr.

In some examples, at least a portion of each of support wires 22 is embedded in wall 32. For example, exterior layer 38 defines a plurality of lumens 40. Each of lumens 40 may open to an exterior surface 13 of elongate member 12 at respective distal openings 30 (FIG. 2A). Lumens 40 are sized to enable support wires 22 to slide in the proximal-distal direction relative to elongate member 12, e.g., relative to proximal constraint 24. In this way, support wires 22 may be advanced distally from lumens 40 to cause at least a portion of support wires 22 between distal constraint 24 and proximal constraint 28 to expand radially outward from exterior surface 13 of elongate member 12. In some examples, a surface of lumens 40 may include a lubricous coating selected to reduce friction, such as, for example, polytetrafluoroethylene (PTFE), between exterior layer 38 and support wire 22 or otherwise facilitate sliding of support wires 22 within lumens 40. In this way, support wires 22 are advanceable relative to elongate member 12.

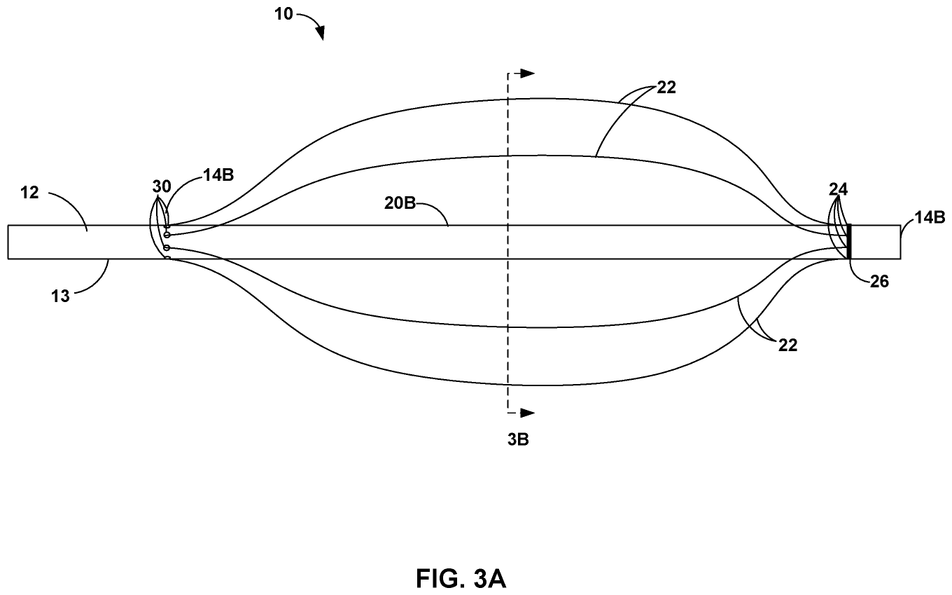

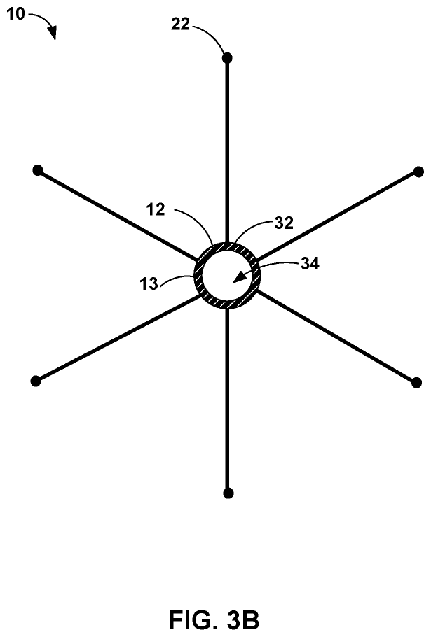

FIG. 3A is a conceptual diagram illustrating distal portion 20B of elongate member 12 with support wires 22 in an deployed configuration. When in the deployed configuration in a blood vessel, support wires 22 may enable blood flow through the blood vessel past the radially expanded portion of support wires 22 (e.g., in the space between adjacent wires 22 and between the inner surfaces of wires 22 closest to and facing elongated member 12 and an outer surface of elongated member 12), whereas other support structures, such as, for example, a balloon structure, may restrict or block blood flow. Enabling blood flow past the expanded portion of support wires 22 may be beneficial in some medical procedures, e.g., it may enable a clinician to keep support wires 22 radially expanded in the vasculature for a longer period of time.

In the deployed configuration, support wire 22 may be expanded radially away from elongate member 12 and may be configured to engage tissue with the vasculature of a patient. For example, when in the deployed configuration, support wires 22 are advanced from distal openings 30. As support wires 22 advance from distal openings 30, supports wires 22 expand radially outward, e.g., away from exterior surface 13 of elongate member 12. As support wires 22 expand radially outward, one or more portions of one or more support wires 22 may contact and engage a vessel wall within the vasculature of a patient. The contact between support wires 22 and the vessel wall may help stabilize the position of distal tip 14B, may help position elongate member 12 away from the vasculature wall to aid in navigation of elongate member 12 through vasculature, or otherwise provide backup support to catheter 10. In this way, catheter 10 may engage with support wires 22 tissue within the vasculature of a patient to provide backup support for catheter 10.

FIG. 3B is a conceptual diagram illustrating a cross section (cross section 3B as indicated in FIG. 3A) of distal portion 20B of elongate member 12 with support wires 22 in an deployed configuration. As discussed above, elongate member 12 includes a wall 32 defining exterior surface 13 and lumen 34. As illustrated in FIG. 3B, in some examples, support wires 22 are substantially evenly spaced about a central axis (e.g., longitudinal axis 15, FIG. 1). In other examples, support wires 22 may be unevenly spaced about the central axis. As discussed above, the spacing of support wires 22 may be selected to control an amount of backup support for a selected side of catheter 10.

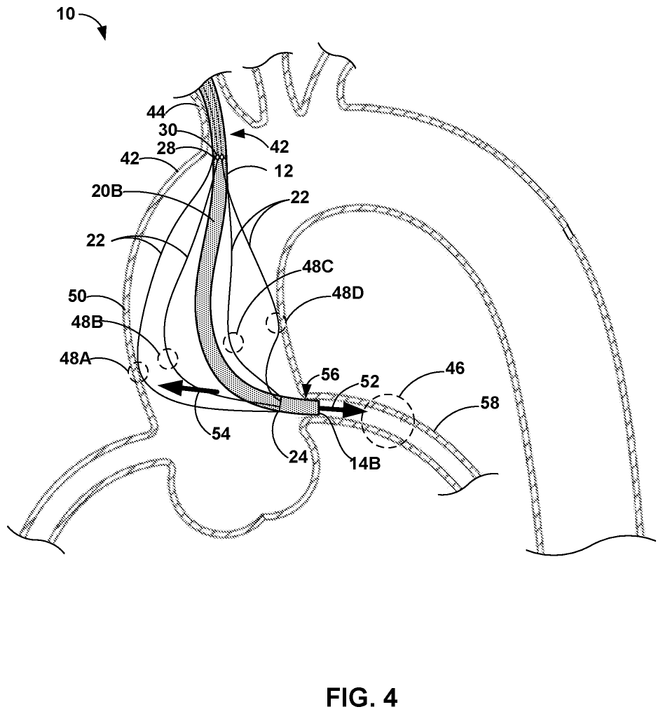

FIG. 4 is a conceptual diagram illustrating distal portion 20B of elongate member 12 with support wires 22 in a deployed configuration engaging the walls of a blood vessel of a patient. In the example of FIG. 4, catheter 10 is introduced into the vasculature of the patient via the right radial artery (e.g., a right radial approach), and guided through the brachiocephalic artery 44 and ostium 42 of the aortic arch to a target treatment site 46. The target treatment site 46 includes a portion of a coronary artery that includes a calcified lesion, e.g., calcified plaque buildup, stenosis, aneurysm, or other diseased area. As discussed above, accessing a coronary artery, e.g., coronary artery 58, from a right radial approach, may lack a sufficient number of turns and natural backup support options to establish a sufficient amount of backup support for catheter 10 from the anatomy of the patient. The example of FIG. 4 illustrates one approach to particular target site, however, catheter 10 may be used in the same or different approaches to the same or different target sites.

As illustrated in FIG. 4, when in the deployed configuration, each support wire of support wires 22 may contact a portion of the ascending aorta 50 at a respective contact point 48A,48B, 48C, and 48D (collectively, “contact point 48”). For example, a clinician may actuate controls on or near hub assembly 16 (FIG. 1) to advance support wires 22 from distal openings 30. Advancing support wires 22 from distal openings 30 may cause support wires 22 to expand radially outward from elongate member 12 and contact the surrounding vessel wall, e.g., ascending aorta 50, to establish additional backup support. As discussed above, support wires 22 may be actuatable (controlled) individually or in groups and/or evenly or unevenly spaced around a perimeter of elongate member 12. In this way, catheter 10 enable selective expansion of support wires 22, which may enable the clinician to select subsets of wires 22 (e.g., all the support wires 22 or only some of the wires 22) to help guide catheter 10 in different directions relative to the vasculature of the patient, provide backup support at selected sides of catheter 10, and/or to enable distal portion 20B of catheter 10 to have a selected curve within the vasculature, which may help with navigation around particular curves in the vasculature. In some examples, the backup support enables by support sires 22 may help anchor elongate member 12 within a blood vessel, which may help reduce the movement of catheter 10, e.g., distal portion 20B, within the vasculature of the patient, particularly during the advancement of a treatment device through lumen 34 of elongate member 12 towards target treatment site 46.

For example, a treatment device may be deployed from distal tip 14B of catheter 10 in the direction of arrow 52 toward target treatment site 46. Deployment of the treatment device from distal tip 14B cause an axial force in the opposite direction of deployment along the catheter as indicated by arrow 54. For example, when a treatment device is forced by the clinician distally relative to distal tip 14B and further into the vasculature or lesion of the patient, the resistance encountered by the treatment device within the vessel can sometimes transfer the force into catheter 10, e.g., distal portion 20B. The resistance may result in an axial force on distal portion 20B in the proximal direction generally along longitudinal axis 15, as indicated by arrow 54, rather than forcing the treatment device further into the vessel of the patient. By deploying support wires 22 in the vasculature, e.g., ascending aorta 50, the axial force may be transferred to the vessel wall rather than causing distal portion 20B to bend or become dislodged from ostium 56 of coronary artery 58.

As discussed above, in some examples, support wires 22 may be substantially evenly distributed around an outer perimeter of distal portion 20B of elongate member. The even distribution of support wires 22 may enable an clinician to advance support wires 22 such that distal portion 20B in supported near the center of the vessel, e.g., near the center of ascending aorta. Near the center may be a distance greater than direct contact with the vessel wall, such as at least 2 millimeters from the vessel wall, such as at least 5 millimeters from the vessel wall. In some examples, support wires 22 may be unevenly distributed around an outer perimeter of distal portion 20B of elongate member 12. For example, more distal wires may be distributed on a side of elongate member 12 that can be oriented in the direction of the axial force indicated by arrow 54, such that additional support may be provided to oppose the axial force.

In some examples, one or more of support wires 22 may include a preformed curve having an apex that, when in the deployed configuration, is closer to proximal constraint 28 than distal constraint 24. For example, as illustrated in FIG. 4, contact points 48 may be closer to distal tip 14B than distal openings 30. In this way, support wires 22 may provide backup support for distal portion 20B at a location that enables more effective transfer of the axial force to the vessel wall and/or reduces deflection of distal portion 20B compared to support wires 22 that do not include a preformed curve having an apex that is closer to proximal constraint 28 than distal constraint 24.

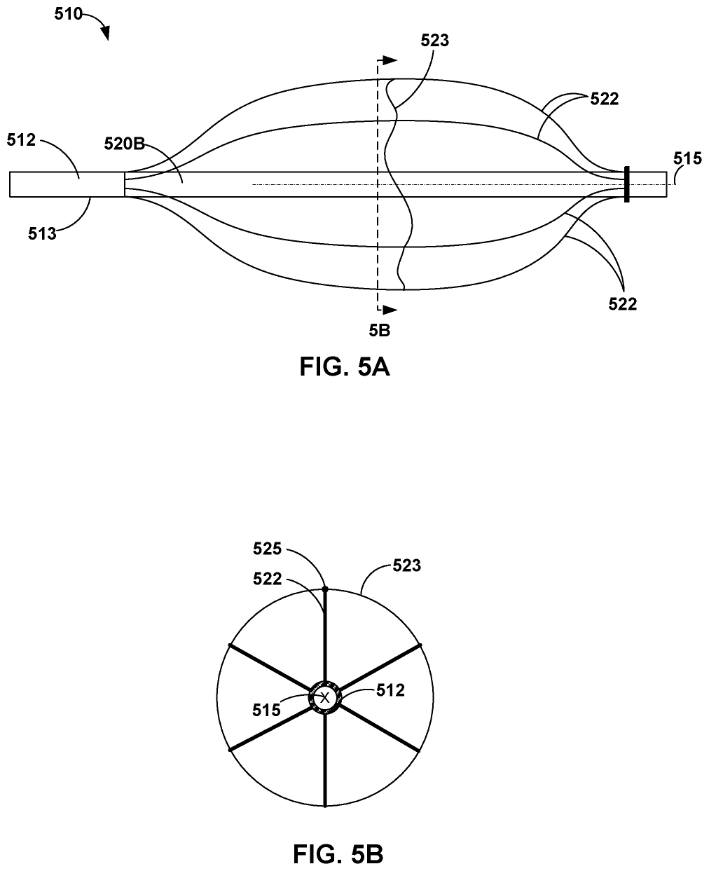

In some examples, at least two support wires of the plurality of support wires are coupled by a common connecting member extending transverse to the longitudinal axis. FIGS. 5A and 5B are conceptual diagrams illustrating a side view and a cross sectional view, respectively, of distal portion 520B of elongate member 512 of an example catheter 510 with support wires 522 and a connecting member 523 in a deployed configuration. Catheter 510 and support wires 522 may be the same as or substantially similar to catheter 10 and support wires 22, respectively, discussed above in reference to FIGS. 1-4, except for the differences described herein. For example, adjacent support wires of support wires 522 are coupled by connecting member 523. When in the deployed configuration, connecting member 523 extends transverse to longitudinal axis 515.

Connecting member 523 is configured to maintain a selected spacing between selected support wires 522 when wires 522 are in the deployed configuration. For example, as illustrated in FIG. 5B, connecting member 523 may be welded, adhered, or otherwise fixed to selected support wires 522 at a respective joint 525. In some examples, connecting member 523 may be fixed to each support wire 522 such that connecting member 523 may maintain a selected spacing between each adjacent support wire 522. In some examples, connecting member 523 may be fixed to fewer than all support wires. For example, connecting member 523 may be fixed to every other support wire 522, or any other number of support wires 522. Although illustrated as traversing the circumference of support wires 522 in the deployed configuration, in other examples, connecting member 523 may traverse fewer than all support wires 522. The number and configuration of support wires 522 fixed to connecting member 523 may be selected to control a configuration of support wires 522 in the deployed configuration, including, but not limited to, urging a majority of support wires 522 to a selected region when in the deployed configuration.

In some examples, connecting member 523 may be flexible to enable connecting member 523 to be positioned directed adjacent to elongate member 512. For example, connecting member 523 may lay flat against exterior surface 513 of elongate member 512, when in support wires 522 are in the collapsed configuration. In some examples, connecting member 523 may include a shape memory alloy, such as a nickel titanium alloy, or a flexible medical polymer configured to have a preformed collapsed configuration in which support wires 522 and connecting member 523 lay substantially flat against exterior surface 513 of elongate member 512 and a deployed configuration as illustrated in FIGS. 5A and 5B.

In some examples, catheter 510 may include a plurality of connecting members 523. For example, the plurality of connecting members and the support wires 522 may define a wire mech structure. By using a wire mesh structure, catheter 510 may have a greater number of contact points with an interior surface of the vasculature of a patient, e.g., a vessel wall, compared to a catheter without a wire mesh structure. The greater number of contact points may increase the support of catheter 510 when the wire mesh structure is in the deployed configuration.

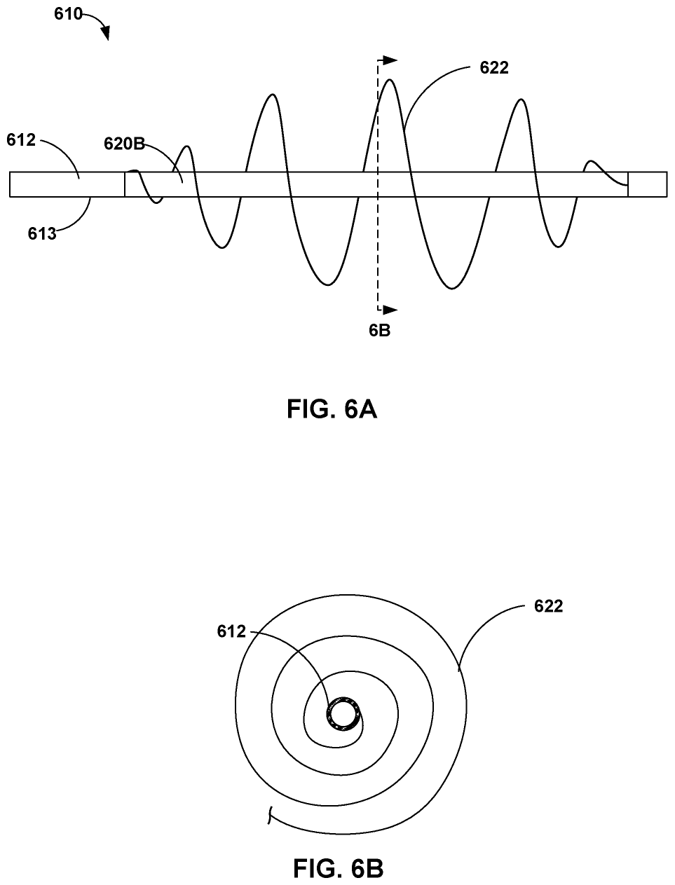

In some examples, a support wire may include at least one wire configured to form a spiral or arc surrounding at least a portion of the elongate member when in the deployed configuration. FIGS. 6A and 6B are conceptual diagrams illustrating a side view and a cross sectional view, respectively, of distal portion 620B of elongate member 612 of another example catheter 610. Catheter 610 may be the same as or substantially similar to catheter 10 described above in reference to FIG. 1-4, except for the differences describe herein. For example, catheter 610 includes spiral support wire 622, which is configured to expand from a collapsed configuration to a deployed configuration (shown in FIG. 6A). In the collapsed configuration, spiral support wire 622 may be wound in a spiral against exterior surface 613 of elongate member 612. In the deployed configuration, support wire 622 forms a spiral surrounding at least a portion of elongate member 612 and expanded radially outward from exterior surface 13 of elongate member 12. Although illustrated as a spiral, support wire 622 may include other shapes such as a semicircle. Moreover, the shape of the deployed support wire 622 may include any suitable shape such as a frustum, a sphere, a cone, or a cylinder. When in the deployed configuration, one or more regions of the spiral may form the contact point with the vessel wall to support catheter 610. When in the collapsed configuration, the spiral may tighten about elongate member 612 such that support wire 622 lays substantially flat against exterior surface 613 of elongate member 612.

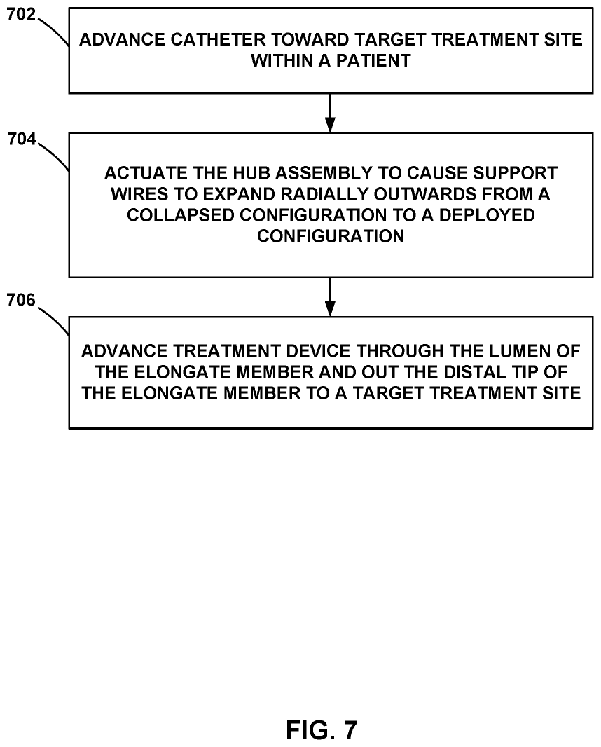

The catheters describe herein may be used to deliver a treatment device to a target treatment site within a vasculature of a patient using any suitable technique. FIG. 7 is a flow diagram illustrating an example method of delivering a treatment device to a target treatment site within a vasculature of a patient using an example catheter. The catheter may be the same as or substantially similar to catheters 10, 510, and/or 610 discussed above with respect to FIGS. 1-6B. Although FIG. 7 is described with respect to catheter 10, in other examples, the method of FIG. 7 may be used with other catheters having support wires configured to provide backup support of a distal portion of the catheter.

The technique illustrated in FIG. 7 includes advancing catheter 10 through vasculature toward target treatment site 46 within a patient. In some examples, when advancing catheter 10 toward target treatment site 46 within the patient, support wires 22 are in a collapsed, low-profile configuration and distal portion 21 of each support wire is positioned on an exterior surface of the distal portion 20B of elongate member 12. As discussed above, positioning distal portion 21 of support wires 22 on exterior surface 13 of distal portion 20B to define a relatively smooth surface may improve the navigability of distal tip 14B through the vasculature of the patient by reducing any resistance generated between the inner wall of the vessel and exterior surface 13 or support wires 22.

The technique illustrated in FIG. 7 includes actuating hub assembly 16, e.g., control members 17, to cause support wires 22 to expand radially outwards from a collapsed configuration to a deployed configuration. Distal portion 21 of at least one of support wire 22 may be configured to engage with a vessel wall, e.g., an interior wall of ascending aorta 50, in the deployed configuration. In examples in which elongate member 12 includes wall 32 defining lumen 34 and at least a portion of each of support wires 22 is embedded in wall 32, actuating hub assembly 16, e.g., control members 17, may cause distal portion 21 of each of support wires 22 to advance through wall 32 at distal opening 30 on distal portion 20B of elongate member 12.

In examples in which hub assembly 16 includes control members 17 operatively coupled support wires 22, the technique may include actuating control member 17 to control support wires 22 between the collapsed configuration and the deployed configuration. In some examples, each of control member 17 may be actuated to control one or more support wires between the collapsed configuration and the deployed configuration. For example, each respective control member 17 may be operative coupled to a respective support wire 22 such that actuating control members 17 includes actuating each of control members 17 to expand each respective support wire 22 between the collapsed configuration to the deployed configuration.

In examples, in which support wires 22 include a radiopaque material, the technique may include, when advancing the catheter toward the target treatment site or actuating hub assembly 16 to control support wires 22 from the collapsed configuration to the deployed configuration, visualizing a position of at least one support wire 22 using fluoroscopy. In examples in which distal constraint 24 includes radiopaque marker band 26, the technique may include, when advancing the catheter toward the target treatment site, visualizing a position of distal constraint 24 using fluoroscopy. Although described as using fluoroscope, in some examples, other medical imaging techniques may be used to visualize one or more portions of catheter 10, such as, for example, ultrasound, x-ray, or the like. Visualizing one or more portions catheter 10 may enable more precise positioning of catheter 10 with respect to anatomical structures within the vasculature of the patient to improve backup support using support wires 22 and/or improve positioning of catheter 10 with respect to target treatment site 46.

The technique illustrated in FIG. 7 includes advancing a treatment device through the lumen of the elongate member and out the distal tip of the elongate member to a target treatment site. After advancing the treatment device to target treatment site 46, the technique may includes performing a treatment procedure, such as, for example, balloon angioplasty, stenting, thrombectomy, atherectomy, or other interventional procedures. After performing the treatment, the treatment device may be withdrawn into catheter 10, and catheter 10 removed from the vasculature of the patient.

The following clauses illustrate example subject matter described herein.

Clause 1. A catheter comprising: an elongate member extending along a longitudinal axis from a proximal end to a distal tip, the elongate member defining an inner lumen and a distal opening to the inner lumen; and a plurality of support wires extending along at least a distal portion of the elongate member, wherein the plurality of support wires is slidably engaged with a proximal constraint, wherein a distal end of each support wire of the plurality of support wires is attached to a distal constraint, wherein the plurality of support wires is configured to expand radially outwards between the proximal and distal constraints from a collapsed configuration to a deployed configuration, and wherein, when in the deployed configuration, a section of at least one support wire of the plurality of support wires between the proximal and distal constraints is configured to engage with a vessel wall to position the elongate member away from the vessel wall.

Clause 2. The catheter of clause 1, wherein the elongate member comprises a wall defining a plurality of wire lumens, each wire lumen terminating at a respective distal opening of a plurality of distal openings, wherein at least a portion of each support wire of the plurality of support wires extends through a respective wire lumen of the plurality of wire lumens, and wherein the section of each support wire of the plurality of support wires protrudes through the wall at the respective distal opening of the plurality of distal openings.

Clause 3. The catheter of clause 1 or 2, wherein the support wires of the plurality of support wires are evenly distributed around an outer perimeter of the distal portion of the elongate member.

Clause 4. The catheter of clause 1 or 2, wherein the support wires of the plurality of support wires is unevenly distributed around an outer perimeter of the distal portion of the elongate member.

Clause 5. The catheter of any one of clauses 1 through 4, wherein the distal end of each support wire of the plurality of support wires is about 1 millimeter to about 10 centimeters proximal to the distal tip of the elongate member.

Clause 6. The catheter of any one of clauses 1 through 5, wherein, when the plurality of support wires is in the collapsed configuration, the section of each support wire is positioned on an exterior surface of the distal portion of the elongate member.

Clause 7. The catheter of any one of clauses 1 through 6, wherein, when the plurality of support wires is in the deployed configuration, the section of the at least one support wire defines a preformed curve having an apex that is closer to the proximal constraint than the distal constraint.

Clause 8. The catheter of any one of clauses 1 through 7, wherein at least two support wires of the plurality of support wires are coupled by a connecting member extending transverse to the longitudinal axis.

Clause 9. The catheter of any one of clauses 1 through 8, wherein the plurality of support wires comprises a wire mesh structure.

Clause 10. The catheter of any one of clauses 1 through 9, wherein the plurality of support wires comprises at least one wire configured to form a spiral or arc surrounding at least a portion of the elongate member when in the deployed configuration.

Clause 11. The catheter of any one of clauses 1 through 10, wherein at least two support wires of the plurality of support wires are coupled to a single control member extending from the proximal end of the elongate member to the distal portion of the elongate member.

Clause 12. The catheter of any one of clauses 1 through 11, wherein the plurality of support wires comprises a shape memory alloy or a nickel titanium alloy.

Clause 13. The catheter of any one of clauses 1 through 12, wherein the plurality of support wires comprises a radiopaque material.

Clause 14. The catheter of any one of clauses 1 through 13, wherein the distal constraint comprises a radiopaque marker band.

Clause 15. The catheter of any one of clauses 1 through 14, wherein each support wire of the plurality of support wires is individually actuatable into the deployed configuration.

Clause 16. The catheter of any one of clauses 1 through 15, further comprising a hub assembly at the proximal end of the elongate member, wherein the hub assembly comprises a control member operatively coupled the plurality of support wires, wherein the control member is configured to control the plurality of support wires between the collapsed configuration and the deployed configuration.

Clause 17. A medical assembly comprising: a catheter comprising: a hub assembly;

an elongate member extending along a longitudinal axis from a proximal end coupled to the hub assembly to a distal tip, the elongate member defining an inner lumen and a distal opening to the inner lumen; and a plurality of support wires extending along at least a distal portion of the elongate member, wherein the plurality of support wires is slidably engaged with a proximal constraint, wherein a distal end of each support wire of the plurality of support wires is attached to a distal constraint, wherein the plurality of support wires is configured to expand radially outwards between the proximal and distal constraints from a collapsed configuration to a deployed configuration, and wherein, when in the deployed configuration, a section of at least one support wire of the plurality of support wires between the proximal and distal constraints is configured to engage with a vessel wall in the deployed configuration; and a treatment device configured to be received in the inner lumen of the elongate member.

Clause 18. The medical assembly of clause 17, wherein the elongate member comprises a wall defining a plurality of wire lumens, each wire lumen terminating at a respective distal opening of a plurality of distal openings, wherein at least a portion of each support wire of the plurality of support wires extends through a respective wire lumen of the plurality of wire lumens, and wherein the section of each support wire of the plurality of support wires protrudes through the wall at the respective distal opening of the plurality of distal openings.

Clause 19. The medical assembly of clause 17 or 18, wherein the support wires of the plurality of support wires are evenly distributed around an outer perimeter of the distal portion of the elongate member.

Clause 20. The medical assembly of clause 17 or 18, wherein the support wires of the plurality of support wires are unevenly distributed around an outer perimeter of the distal portion of the elongate member.

Clause 21. The medical assembly of any one of clauses 17 through 20, wherein the distal end of each support wire of the plurality of support wires is about 1 millimeter to about 10 centimeters proximal to the distal tip of the elongate member.

Clause 22. The medical assembly of any one of clauses 17 through 21, wherein, when the plurality of support wires is in the collapsed configuration, the distal portion of each support wire is positioned on an exterior surface of the section of the elongate member.

Clause 23. The medical assembly of any one of clauses 17 through 22, wherein, when the plurality of support wires is in the collapsed configuration, the section of the at least one support wire defines a preformed curve having an apex that is closer to the proximal constraint than the distal constraint.

Clause 24. The medical assembly of any one of clauses 17 through 23, wherein at least two support wires of the plurality of support wires are coupled by a connecting member extending transverse to the longitudinal axis.

Clause 25. The medical assembly of any one of clauses 17 through 24, wherein the plurality of support wires comprises a wire mesh structure.

Clause 26. The medical assembly of any one of clauses 17 through 25, wherein the plurality of support wires comprises at least one wire configured to form a spiral or arc surrounding at least a portion of the elongate member when in the deployed configuration.

Clause 27. The medical assembly of any one of clauses 17 through 26, wherein at least two support wires of the plurality of support wires are coupled to a single control member extending from the proximal end of the elongate member to the distal portion of the elongate member.

Clause 28. The medical assembly of any one of clauses 17 through 27, wherein the plurality of support wires comprises a shape memory alloy or a nickel titanium alloy.

Clause 29. The medical assembly of any one of clauses 17 through 28, wherein the plurality of support wires comprises a radiopaque material.

Clause 30. The medical assembly of any one of clauses 17 through 29, wherein the distal constraint comprises a radiopaque marker band.

Clause 31. The medical assembly of any one of clauses 17 through 30, wherein each support wire of the plurality of support wires is individually actuatable into the deployed configuration.

Clause 32. The medical assembly of any one of clauses 17 through 31, wherein the hub assembly comprises a control member operatively coupled the plurality of support wires, wherein the control member is configured to control the plurality of support wires between the collapsed configuration and the deployed configuration.

Clause 33. A method comprising: advancing a catheter through vasculature of a patient, wherein the catheter comprises: an elongate member extending along a longitudinal axis from a proximal end to a distal tip, the elongate member defining an inner lumen and a distal opening to the inner lumen; and a plurality of support wires extending along at least a distal portion of the elongate member, wherein the plurality of support wires is slidably engaged with a proximal constraint, and wherein a distal end of each support wire of the plurality of support wires is attached to a distal constraint; actuating the plurality of support wires to expand radially outwards between the proximal and distal constraints from a collapsed configuration to a deployed configuration, wherein, when in the deployed configuration, a section of at least one support wire of the plurality of support wires between the proximal and distal constraints is configured to engage with a vessel wall to position the elongate member away from the vessel wall; advancing a treatment device through the inner lumen of the elongate member to a target treatment site.

Clause 34. The method of clause 33, wherein the elongate member comprises a wall defining a plurality of wire lumens, each wire lumen terminating at a respective distal opening of a plurality of distal openings, wherein at least a portion of each support wire of the plurality of support wires extends through a respective wire lumen of the plurality of wire lumens, and wherein the section of each support wire of the plurality of support wires protrudes through the wall at the respective distal opening of the plurality of distal openings.

Clause 35. The method of clause 33 or 34, wherein the support wires of the plurality of support wires are evenly distributed around an outer perimeter of the distal portion of the elongate member.

Clause 36. The method of clause 33 or 34, wherein the support wires of the plurality of support wires is unevenly distributed around an outer perimeter of the distal portion of the elongate member.

Clause 37. The method of any one of clauses 33 through 36, wherein the distal end of each support wire of the plurality of support wires is about 1 millimeter to about 10 centimeters proximal to the distal tip of the elongate member.

Clause 38. The method of any one of clauses 33 through 37, wherein, when advancing the catheter through the vasculature, the section of each support wire is positioned on an exterior surface of the distal portion of the elongate member.

Clause 39. The method of any one of clauses 33 through 38, wherein, when the plurality of support wires is in the deployed configuration, at section of the least one support wire defines a preformed curve having an apex that is closer to the proximal constraint than the distal constraint.

Clause 40. The method of any one of clauses 33 through 39, wherein at least two support wires of the plurality of support wires are coupled by a connecting member extending transverse to the longitudinal axis.

Clause 41. The method of any one of clauses 33 through 40, wherein the plurality of support wires comprises a wire mesh structure.

Clause 42. The method of any one of clauses 33 through 41, wherein the plurality of support wires comprises at least one wire configured to form a spiral or arc surrounding at least a portion of the elongate member when in the deployed configuration.

Clause 43. The method of any one of clauses 33 through 42, wherein at least two support wires of the plurality of support wires are coupled to a single control member extending from the proximal end of the elongate member to the distal portion of the elongate member.

Clause 44. The method of any one of clauses 33 through 43, wherein the plurality of support wires comprises a shape memory alloy or a nickel titanium alloy.

Clause 45. The method of any one of clauses 33 through 44, wherein the catheter further comprises a hub assembly at the proximal end of the elongate member, wherein the hub assembly comprises a control member operatively coupled the plurality of support wires, and wherein actuating the plurality of support wires comprises actuating the control member to control the plurality of support wires between the collapsed configuration and the deployed configuration.

Clause 46. The method of clause 45, wherein the control member comprises a plurality of control members, each respective control member operative coupled to a respective support wire of the plurality of support wires, wherein actuating the control member comprises actuating each of the plurality of control members to expand each respective support wire of the plurality of support wires between the collapsed configuration to the deployed configuration.

Clause 47. The method of any one of clauses 33 through 46, wherein the plurality of support wires comprises a radiopaque material, wherein the method further comprises visualizing a position of at least one support wire of the plurality of support wires using fluoroscopy.

Clause 48. The method of any one of clauses 33 through 47, wherein the distal constraint comprises a radiopaque marker band, wherein the method further comprises, when advancing the catheter toward the target treatment site, visualizing a position of the distal constraint using fluoroscopy.

Clause 49. The method of any one of clauses 33 through 48, wherein advancing the catheter through the vasculature of the patient comprises advancing the catheter through a radial artery of the patient.

Clause 50. The method of clause 49, wherein the radial artery comprises a right radial artery of the patient.

Various examples have been described. These and other examples are within the scope of the following claims.

What is claimed is:

1. A catheter comprising:
an elongate member extending along a longitudinal axis from a proximal end to a distal tip, the elongate member defining an inner lumen and a distal opening to the inner lumen; and
a plurality of support wires extending along at least a distal portion of the elongate member,
wherein the plurality of support wires is slidably engaged with a proximal constraint,
wherein a distal end of each support wire of the plurality of support wires is attached to a distal constraint,
wherein the plurality of support wires is configured to expand radially outwards between the proximal and distal constraints from a collapsed configuration to a deployed configuration, and
wherein, when in the deployed configuration, a section of at least one support wire of the plurality of support wires between the proximal and distal constraints is configured to engage with a vessel wall to position the elongate member away from the vessel wall.

2. The catheter of claim 1, wherein the elongate member comprises a wall defining a plurality of wire lumens, each wire lumen terminating at a respective distal opening of a plurality of distal openings, wherein at least a portion of each support wire of the plurality of support wires extends through a respective wire lumen of the plurality of wire lumens, and wherein the section of each support wire of the plurality of support wires protrudes through the wall at the respective distal opening of the plurality of distal openings.

3. The catheter of claim 1, wherein the support wires of the plurality of support wires are evenly distributed around an outer perimeter of the distal portion of the elongate member.

4. The catheter of claim 1, wherein the support wires of the plurality of support wires is unevenly distributed around an outer perimeter of the distal portion of the elongate member.

5. The catheter of claim 1, wherein the distal end of each support wire of the plurality of support wires is about 1 millimeter to about 10 centimeters proximal to the distal tip of the elongate member.

6. The catheter of claim 1, wherein, when the plurality of support wires is in the collapsed configuration, the section of each support wire is positioned on an exterior surface of the distal portion of the elongate member.

7. The catheter of claim 1, wherein, when the plurality of support wires is in the deployed configuration, the section of the at least one support wire defines a preformed curve having an apex that is closer to the proximal constraint than the distal constraint.

8. The catheter of claim 1, wherein at least two support wires of the plurality of support wires are coupled by a connecting member extending transverse to the longitudinal axis.

9. The catheter of claim 1, wherein the plurality of support wires comprises a wire mesh structure.

10. The catheter of claim 1, wherein the plurality of support wires comprises at least one wire configured to form a spiral or arc surrounding at least a portion of the elongate member when in the deployed configuration.

11. The catheter of claim 1, wherein at least two support wires of the plurality of support wires are coupled to a single control member extending from the proximal end of the elongate member to the distal portion of the elongate member.

12. The catheter of claim 1, wherein the plurality of support wires comprises a shape memory alloy or a nickel titanium alloy.

13. The catheter of claim 1, wherein the plurality of support wires comprises a radiopaque material.

14. The catheter of claim 1, wherein the distal constraint comprises a radiopaque marker band.

15. The catheter of claim 1, wherein each support wire of the plurality of support wires is individually actuatable into the deployed configuration.

16. The catheter of claim 1, further comprising a hub assembly at the proximal end of the elongate member, wherein the hub assembly comprises a control member operatively coupled the plurality of support wires, wherein the control member is configured to control the plurality of support wires between the collapsed configuration and the deployed configuration.

17. A medical assembly comprising
a catheter comprising:
a hub assembly;
an elongate member extending along a longitudinal axis from a proximal end coupled to the hub assembly to a distal tip, the elongate member defining an inner lumen and a distal opening to the inner lumen; and
a plurality of support wires extending along at least a distal portion of the elongate member,
wherein the plurality of support wires is slidably engaged with a proximal constraint,
wherein a distal end of each support wire of the plurality of support wires is attached to a distal constraint,
wherein the plurality of support wires is configured to expand radially outwards between the proximal and distal constraints from a collapsed configuration to a deployed configuration, and
wherein, when in the deployed configuration, a section of at least one support wire of the plurality of support wires between the proximal and distal constraints is configured to engage with a vessel wall in the deployed configuration; and
a treatment device configured to be received in the inner lumen of the elongate member.

18. The medical assembly of claim 17, wherein the elongate member comprises a wall defining a plurality of wire lumens, each wire lumen terminating at a respective distal opening of a plurality of distal openings, wherein at least a portion of each support wire of the plurality of support wires extends through a respective wire lumen of the plurality of wire lumens, and wherein the section of each support wire of the plurality of support wires protrudes through the wall at the respective distal opening of the plurality of distal openings.

19. The medical assembly of claim 17, wherein the support wires of the plurality of support wires are evenly distributed around an outer perimeter of the distal portion of the elongate member.

20. The medical assembly of claim 17, wherein the support wires of the plurality of support wires are unevenly distributed around an outer perimeter of the distal portion of the elongate member.

21. The medical assembly of claim 17, wherein the distal end of each support wire of the plurality of support wires is about 1 millimeter to about 10 centimeters proximal to the distal tip of the elongate member.

22. The medical assembly of claim 17, wherein, when the plurality of support wires is in the collapsed configuration, the distal portion of each support wire is positioned on an exterior surface of the section of the elongate member.

23. The medical assembly of claim 17, wherein, when the plurality of support wires is in the collapsed configuration, the section of the at least one support wire defines a preformed curve having an apex that is closer to the proximal constraint than the distal constraint.

24. The medical assembly of claim 17, wherein at least two support wires of the plurality of support wires are coupled by a connecting member extending transverse to the longitudinal axis.

25. The medical assembly of claim 17, wherein the plurality of support wires comprises a wire mesh structure.

26. The medical assembly of claim 17, wherein the plurality of support wires comprises at least one wire configured to form a spiral or arc surrounding at least a portion of the elongate member when in the deployed configuration.

27. The medical assembly of claim 17, wherein at least two support wires of the plurality of support wires are coupled to a single control member extending from the proximal end of the elongate member to the distal portion of the elongate member.

28. The medical assembly of claim 17, wherein each support wire of the plurality of support wires is individually actuatable into the deployed configuration.

29. The medical assembly of claim 17, wherein the hub assembly comprises a control member operatively coupled the plurality of support wires, wherein the control member is configured to control the plurality of support wires between the collapsed configuration and the deployed configuration.

30. A method comprising:
advancing a catheter through vasculature of a patient, wherein the catheter comprises:
an elongate member extending along a longitudinal axis from a proximal end to a distal tip, the elongate member defining an inner lumen and a distal opening to the inner lumen; and
a plurality of support wires extending along at least a distal portion of the elongate member, wherein the plurality of support wires is slidably engaged with a proximal constraint, and wherein a distal end of each support wire of the plurality of support wires is attached to a distal constraint;
actuating the plurality of support wires to expand radially outwards between the proximal and distal constraints from a collapsed configuration to a deployed configuration, wherein, when in the deployed configuration, a section of at least one support wire of the plurality of support wires between the proximal and distal constraints is configured to engage with a vessel wall to position the elongate member away from the vessel wall;
advancing a treatment device through the inner lumen of the elongate member to a target treatment site.

31. The method of claim 30, wherein the catheter further comprises a hub assembly at the proximal end of the elongate member, wherein the hub assembly comprises a control member operatively coupled the plurality of support wires, and wherein actuating the plurality of support wires comprises actuating the control member to control the plurality of support wires between the collapsed configuration and the deployed configuration.

32. The method of claim 31, wherein the control member comprises a plurality of control members, each respective control member operative coupled to a respective support wire of the plurality of support wires, wherein actuating the control member comprises actuating each of the plurality of control members to expand each respective support wire of the plurality of support wires between the collapsed configuration to the deployed configuration.
